(19) United States
(12) Patent Application Publication
(10) Pub. No.: US 2014/0243400 A1
(43) Pub. Date: Aug. 28, 2014

(54) COMPOSITIONS COMPRISING CITRIC ACID AND MALIC ACID AND METHODS AND USES THEREOF

(71) Applicant: University of Alaska Anchorage, Anchorage, AK (US)

(72) Inventor: Colin McGill, Anchorage, AK (US)

(21) Appl. No.: 14/192,681

(22) Filed: Feb. 27, 2014

Related U.S. Application Data
(60) Provisional application No. 61/770,764, filed on Feb. 28, 2013.

(51) Int. Cl. A61K 31/385; A61K 31/198; A61K 45/06; A61K 31/194

(52) U.S. Cl. CPC A61K 31/385; A61K 31/194; A61K 31/198; A61K 45/06
USPC 514/440

(57) ABSTRACT

The invention relates to compositions comprising at least about 500 mg of citric acid; at least about 500 mg of malic acid; and at least about 100 mg of N-acetylcysteine, or at least about 100 mg of α-lipoic acid, or a mixture thereof and methods for use of the compositions thereof. This abstract is intended as a scanning tool for purposes of searching in the particular art and is not intended to be limiting of the present invention.

### CROSS-REFERENCE TO RELATED APPLICATIONS

This Application claims the benefit of U.S. Provisional Application No. 61/770,764, filed on Feb. 28, 2013, which is incorporated herein by reference in its entirety.

### GOVERNMENT LICENSE RIGHTS

This invention was made with government support under USDA grant 2005-34495-16519 awarded by the United States Department of Agriculture. The United States government has certain rights in the invention.

### BACKGROUND

Neurodegenerative diseases and loss of cognitive functions such as memory, attention span, and problem solving are prevalent problems in today's society. Age is the one common risk factor among the myriad of neurodegenerative conditions, and as people age, their chances of developing these conditions increases. Likewise, a patient with a neurodegenerative disease tends to decline in health and faculties as the condition progresses. Additionally, while the cognitive difficulties that are associated with dementia are more severe in individuals with neurodegenerative conditions like Alzheimer's Disease, Parkinson's Disease, Huntington's Disease or Amyotrophic lateral sclerosis, otherwise healthy people often experience some degree of these symptoms as they age. Memory loss and reduced cognitive faculties are also associated with traumatic brain injury (TBI) and stroke, resulting from reactive oxygen species (ROS)-induced neuronal injury resulting in programmed cell death (apoptosis). Moreover, debilitating levels of neurodegeneration are also associated with other common conditions including obesity, diabetes, and non-alcoholic steatohepatitis.

Accordingly, compounds, compositions, and methods that counteract these conditions are highly desired. Such compounds, compositions, and methods are described herein.

### SUMMARY

In accordance with the purpose(s) of the invention, as embodied and broadly described herein, the invention, in one aspect, relates to compositions and methods of use of a composition comprising citric acid and malic acid and further comprising α-lipoic acid or N-acetylcysteine, or a mixture thereof.

Disclosed herein is a composition comprising a) at least about 500 mg of citric acid and b) at least about 500 mg of malic acid and c) at least about 100 mg of N-acetylcysteine or 100 mg α-lipoic acid or a mixture thereof.

Also disclosed herein is a composition comprising citric acid in an amount greater than 10 wt. %, malic acid in an amount greater than 10 wt. %, and N-acetylcysteine or α-lipoic acid or a mixture thereof in an amount greater than 0.5 wt. %.

Also disclosed herein is a dosage form comprising a composition comprising a) at least about 500 mg of citric acid, b) at least about 500 mg of malic acid and c) at least about 100 mg of N-acetylcysteine, or at least about 100 mg of α-lipoic acid, or a mixture thereof for once a day administration.

Also disclosed is a kit comprising a composition comprising a) at least about 500 mg of citric acid, b) at least about 500 mg of malic acid and c) at least about 100 mg of N-acetylcysteine, or at least about 100 mg of α-lipoic acid, or a mixture thereof, and one or more of at least one agent known to increase glutathione, at least one agent know to inhibit activation of nSMase, at least one agent known to decrease levels of neuronal ceramides, or instructions for treating a disorder associated with glutathione dysregulation.

Also disclosed is a composition comprising a) at least about 500 mg of citric acid, b) at least about 500 mg of malic acid and c) at least about 100 mg of N-acetylcysteine, or at least about 100 mg of α-lipoic acid, or a mixture thereof for use as a medicament.

Also disclosed is a composition comprising a) at least about 500 mg of citric acid, b) at least about 500 mg of malic acid and c) at least about 100 mg of N-acetylcysteine, or at least about 100 mg of α-lipoic acid, or a mixture thereof for use in a method for treatment of a neurodegenerative disorder.

Also disclosed is a method of manufacturing a medicament comprising a) at least about 500 mg of citric acid, b) at least about 500 mg of malic acid and c) at least about 100 mg of N-acetylcysteine, or at least about 100 mg of α-lipoic acid, or a mixture thereof with a pharmaceutically acceptable carrier or diluent.

Also disclosed is a method of treating a neurodegenerative disorder associated with glutathione dysregulation in a subject comprising the step of administering to the subject an effective amount of a composition comprising a) at least about 500 mg of citric acid, b) at least about 500 mg of malic acid and c) at least about 100 mg of N-acetylcysteine, or at least about 100 mg of α-lipoic acid, or a mixture thereof, thereby treating the neurodegenerative disorder associated with glutathione dysregulation.

Also disclosed is a method for decreasing the rate of neurodegeneration in a subject comprising the steps of administering to the subject an effective amount of a composition comprising a) at least about 500 mg of citric acid, b) at least about 500 mg of malic acid and c) at least about 100 mg of N-acetylcysteine, or at least about 100 mg of α-lipoic acid, or a mixture thereof, thereby decreasing the rate of neurodegeneration.

Also disclosed is a method for enhancing the memory in a subject comprising the steps of administering to the subject an effective amount of a composition comprising a) at least about 500 mg of citric acid, b) at least about 500 mg of malic acid and c) at least about 100 mg of N-acetylcysteine, or at least about 100 mg of α-lipoic acid, or a mixture thereof, thereby enhancing the memory in the subject.

Also disclosed is a method for increasing glutathione, inhibiting the activation of nSMase and reducing levels of neuronal ceramides in at least one cell comprising contacting the cell with an effective amount of a composition comprising a) at least about 500 mg of citric acid, b) at least about 500 mg of malic acid and c) at least about 100 mg of N-acetylcysteine, or at least about 100 mg of α-lipoic acid, or a mixture thereof, thereby increasing glutathione, inhibiting the activation of nSMase and reducing levels of neuronal ceramides in at least one cell.

Also disclosed is a method for reducing ceramide mediated neurodegeneration in a subject comprising the step of administering to the subject an effective amount of a composition comprising a) at least about 500 mg of citric acid, b) at least about 500 mg of malic acid and c) at least about 100 mg of N-acetylcysteine, or at least about 100 mg of α-lipoic acid, or a mixture thereof, thereby reducing ceramide mediated neurodegeneration in the subject.

Also disclosed herein is a method for reducing ceramide-mediated neurodegeneration, improving learning and memory, and improving neuronal mitochondrial function and insulin sensitivity in a subject comprising administering to the subject an effective amount of a composition comprising a) at least about 500 mg of citric acid, b) at least about 500 mg of malic acid and c) at least about 100 mg of N-acetylcysteine, or at least about 100 mg of α-lipoic acid, or a mixture thereof, thereby reducing ceramide-mediated neurodegeneration, improving learning and memory, and improving neuronal mitochondrial function and insulin sensitivity in the subject.

Also disclosed herein is a method for improving learning in a subject comprising administering to the subject an effective amount of a composition comprising a) at least about 500 mg of citric acid, b) at least about 500 mg of malic acid and c) at least about 100 mg of N-acetylcysteine, or at least about 100 mg of α-lipoic acid, or a mixture thereof, thereby improving learning in the subject.

Also disclosed herein is a method for improving neuronal mitochondrial function in a subject comprising administering to the subject an effective amount of a composition comprising a) at least about 500 mg of citric acid, b) at least about 500 mg of malic acid and c) at least about 100 mg of N-acetylcysteine, or at least about 100 mg of α-lipoic acid, or a mixture thereof, thereby improving neuronal mitochondrial function in the subject.

Also disclosed herein is a method for improving insulin sensitivity in a subject comprising administering to the subject an effective amount of a composition comprising a) at least about 500 mg of citric acid, b) at least about 500 mg of malic acid and c) at least about 100 mg of N-acetylcysteine, or at least about 100 mg of α-lipoic acid, or a mixture thereof, thereby improving insulin sensitivity in the subject.

Also disclosed herein is a composition comprising a) at least about 500 mg of citric acid; b) at least about 500 mg malic acid; c) at least about 100 mg N-acetylcysteine; and d) at least about 100 mg α-lipoic acid.

Also disclosed herein is a composition comprising a) citric acid in an amount greater than 10 wt. %; b) malic acid in an amount greater than 10 wt. %; c) N-acetylcysteine in an amount greater than 0.5 wt. %; and d) α-lipoic acid in an amount greater than 0.5 wt. %.

Also disclosed herein is a composition comprising at least about 500 mg of citric acid and malic acid, wherein both citric acid and malic acid are in amounts greater than about 25 mg. In a further aspect, the composition comprises greater than about 100 mg of N-acetylcysteine or greater than about 100 mg of α-lipoic acid or a mixture thereof.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows Citric acid (CA) and Malic acid (MA) inhibit nSMase-mediate ceramide response to TNF-α. Cytosolic MA and CA metabolism are significant sources of NADPH during periods of redox stress. Elevated cellular pools of MA and CA provide faster reduction of oxidized glutathione (GSSG) to reduced glutathione. Glutathione is an inhibitor of TNF-α mediated nSMase activation. Therefore MA and CA moderate nSMase-mediated ceramide production during proinflammatory events by buffering endogenous redox defense systems.

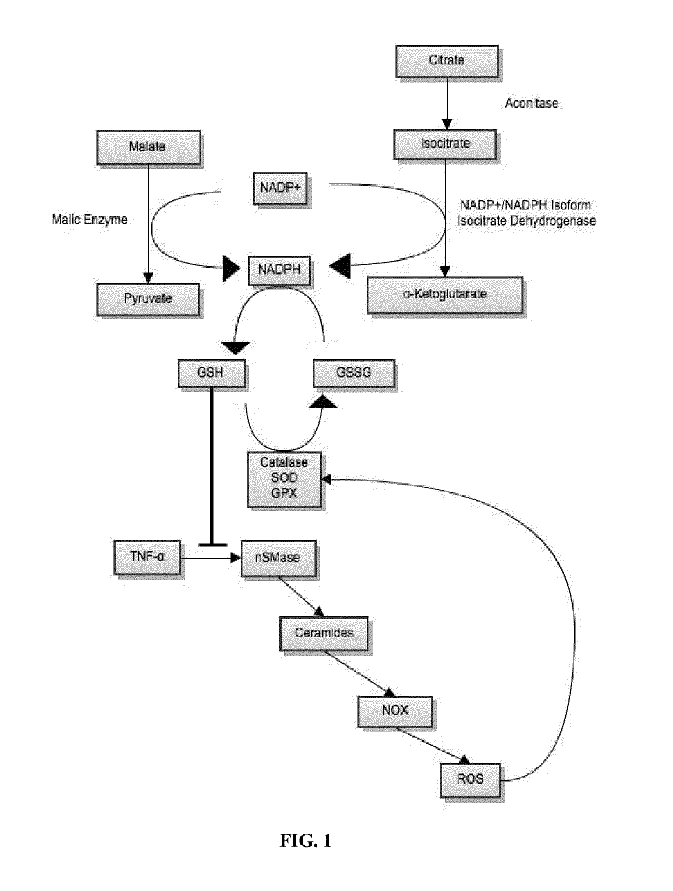

FIG. 2 shows inhibition of nSMase-mediated ceramide release by citrate and malate. Neuroblastomas were not treated (NT) or treated with tumor necrosis factor α (TNF-α), either alone or in conjunction with citrate or malate at 0.5 μg/ml. The results show that both citrate and malate are able to curb nSMase activation.

FIG. 3 shows the mortality effect of citrate and malate. Neither compound demonstrated a cytotoxic effect as neurons were dosed with 0.001-10.0 μg/ml for 24 hours without causing a statistically significant difference in ethidium homodimer staining.

FIG. 4 shows that intracellular concentrations of citrate decrease in response to oxidative insult. Cells were treated with peroxide for 0-90 minutes before being evaluated for citrate content. Results clearly show that as early as 15 minutes citrate decreases by 40%, indicating that it is consumed as part of the endogenous redox defense response.

FIG. 5 shows that intracellular concentrations of malate decrease in response to oxidative insult. Cells were treated with peroxide for 0-90 minutes before being evaluated for malate content. Results clearly show that as early as 30 minutes malate decreases by 20%, indicating that it is consumed as part of the endogenous redox defense response.

FIG. 6 shows that intracellular concentrations of total NADPH transiently decrease in response to oxidative insult. Intracellular NADPH decreases 42% in the first 15 minutes following peroxide treatment before quickly rebounding by the 30 minute mark. After this initial rebound, NADPH continues to decline under sustained redox stress.

FIG. 7 shows intracellular concentrations of total GSH clearly decrease in response to oxidative insult, confirming GSH is actively consumed as part of the endogenous redox defense response in human neurons, specifically to produce regenerate antioxidative enzymes. Total GSH decreased by 50% within the first 15 minutes following treatment with peroxide.

FIG. 8 shows that pretreatment of SH-SY5Y human neuroblastoma cells with malate increases intracellular pools of malate and prevent critical depletion of malate. This indicates that malate supplementation is an effective means of maintaining working pools of this critical component of neuronal redox defense.

FIG. 9 shows that pretreatment of SH-SY5Y human neuroblastoma cells with malate increase intracellular pools of GSH and prevent critical depletion of GSH following redox insult. This indicates that malate supplementation is an effective means of maintaining working pools of GSH for neuronal redox defense.

FIG. 10 shows that oral supplementation of malic acid (200 mg/Kg) in aged F344 rats significantly improves performance in spatial memory. Spatial memory was measured by alternation in a T-maze. The literature shows a young, non-impaired, rat will choose to correctly 85% of the time. Control rats of 10 and 23 months of age, not supplemented with malic acid, showed significant deterioration in spatial memory. Malic acid-supplemented rats in both groups demonstrated statistically significant improvements in spatial memory over controls. Both aged groups supplemented with malic acid performed equivalently to non-impaired rats, at 85% correct alternation, or better.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a dietary supplement,” “an anti-oxidant agent,” or “the subject” includes mixtures of two or more such dietary supplements, anti-oxidant agents, or subjects, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” refers to the target of administration, e.g. an animal. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, fish, bird, or rodent. In one aspect, a subject is a human. The term does not denote a particular age or sex. Thus, adult and newborn subjects, as well as fetuses, whether male or female, are intended to be covered. In one aspect, the subject is a mammal A patient refers to a subject afflicted with a disease or disorder. The term “patient” includes human and veterinary subjects. In some aspects of the disclosed methods, the subject has been diagnosed with a need for treatment of one or more muscle disorders prior to the administering step. In some aspects of the disclosed method, the subject has been diagnosed with a need for promoting muscle health prior to the administering step. In some aspects of the disclosed method, the subject has been diagnosed with a need for promoting muscle health prior, promote normal muscle function, and/or promote healthy aging muscles to the administering step.

As used herein, the term “treatment” refers to the medical management of a patient with the intent to cure, ameliorate, stabilize, or prevent a disease, pathological condition, or disorder. This term includes the use for aesthetic and self-improvement purposes, for example, such uses include, but are not limited to, the administration of the disclosed compound in nutraceuticals, medicinal food, energy bar, energy drink, supplements (such as multivitamins). This term includes active treatment, that is, treatment directed specifically toward the improvement of a disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, fish, bird, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a muscle atrophy disorder” means having been subjected to a physical examination by a person of skill, for example, a physician, and found to have a condition that can be diagnosed or treated by a compound or composition that can promote muscle health, promote normal muscle function, and/or promote healthy aging muscles. As a further example, “diagnosed with a need for promoting muscle health” refers to having been subjected to a physical examination by a person of skill, for example, a physician, and found to have a condition characterized by muscle atrophy or other disease wherein promoting muscle health, promoting normal muscle function, and/or promoting healthy aging muscles would be beneficial to the subject. Such a diagnosis can be in reference to a disorder, such as muscle atrophy, and the like, as discussed herein.

As used herein, the phrase “identified to be in need of treatment for a disorder,” or the like, refers to selection of a subject based upon need for treatment of the disorder. For a subject can be identified as having a need for treatment of a disorder (e.g., a disorder related to muscle atrophy) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “dietary supplement” refers to a compound or composition that is intended to compensate the dietary intake of a subject with regard to specific nutrients, vitamins, minerals, or other dietary components that may be missing or deficient in the subject's normal diet.

As used herein, the term “anti-oxidant agent” refers to a compound, composition, or molecule that is capable of scavenging free radicals, reducing reactive oxygen species or halting oxidative stress chain reactions.

As used herein, the term “neurodegenerative disorder” refers to an umbrella term meant to include a variety of conditions that generally affect the brain. Examples of common neurodegenerative diseases include Alzheimer's Disease (AD), Parkinson's Disease (PD), Huntington's Disease (HD), and Amyotrohpic lateral schlerosis (ALS). These diseases are characterized by progressive deterioration of brain cells, neurons or some subset thereof, ultimately resulting in decreased motor skills or ataxia and/or dementia. The term may also refer to age-related neurodegeneration which occurs naturally in an otherwise healthy subject. Furthermore, the term is also meant to refer to neurodegeneration associated with obesity, diabetes, and non-alcoholic steatohepatitis.

### B. Oxidative Stress and Neurodegeneration

High levels of oxidative stress are associated with neurodegeneration, particularly in diseases like Parkinson's disease, Huntington's disease, Alzheimer's disease, and Amyotrphic lateral sclerosis (ALS) (Anderson, J K. 2004. Nature Reviews Neuroscience, 5; S18-S25). Similarly, oxidative stress is likely one of the underlying causes of age-related neurodegeneration as well (Urano S. et al. 1998. Biofactors, 7(1-2); 103-112; Fukui K. et al. 2001. Ann N Y Acad Sci, 928: 168-175). Oxidative stress is among the leading causes of negative outcomes associated with traumatic brain injury and stroke (Rodriquez-Rodriquez A. et al. 2013. Curr Med Chem, Epub ahead of print; Allen, C. and Bayratutan, U. 2009. Int J Stroke, 4(6); 461-470.)

The brain is particularly susceptible to oxidative stress due to the high metabolic rate of most of the cell types within the brain and the relatively reduced capacity of these cells to regenerate after being damaged (Anderson, J K. 2004. Nature Reviews Neuroscience, 5; S18-S25). Further complicating the matter, neurodegeneration is also accompanied by concomitant decreases in the body's natural ability to alleviate oxidative stress including decreases in the levels of glutathione (Riederer, P. et al. 1989. J. Neurochem. 52, 515-520), the body's most important anti-oxidant enzyme, as well as decreases in glutathione reductase (Pearce, R K. et al. 1997. J. Neural Transm. 104, 661-677), the enzyme responsible for reducing glutathione back to its functional state after it has been oxidized.

As a result, free radicals and reactive oxygen species are able to multiply, causing a dangerous amount of oxidative stress in the brain. This can lead to the hallmarks of neurodegenerative disease including progressive deterioration of brain cells, neurons or some subset thereof, ultimately resulting in decreased motor skills or ataxia and/or dementia.

Inflammation also commonly accompanies neuronal oxidative stress in both disease- and age-mediated neurodegeneration. The cytokine tumor necrosis factor-α (TNF-α) is heavily expressed during inflammatory processes and it elicits the formation of bioactive ceramides by stimulating the activity of neuronal sphingomyelinases (nSMase). Ceramides, in turn, can stimulate further production of reactive oxygen species as well as initiating apoptotic signaling within a cell (Barth B M. et al. 2012. J. Neurosci Res. 90(1); 229-242). Similar mechanisms of ceramide induced neurodegeneration have been shown in obesity, diabetes, and non-alcoholic steatohepatitis (Tong M. et al. 2009. J Alzheimers Dis. 16(4); 705-714).

Disclosed herein is a novel, multifactorial approach to combat age-related neurodegeneration, neurodegenerative disease, and TBI-related neurodegeneration by increasing neuronal glutathione, inhibiting activation of neutral sphingomyelinase (nSMase), and reducing levels of neuronal ceramides via dietary supplementation, pharmacological intervention, or otherwise administering the disclosed compositions. The composition improves neurodegeneration through multiple approaches (a, b, and c below):

a. Administration of malic acid (MA) and citric acid (CA) increase the body's capacity to convert glutathione disulfide (GSSG) to glutathione by increasing intracellular pools of NADPH. Additionally, MA increases levels of mitochondrial ATP through the malate-aspartate shuttle, improving neuronal function. b. α-lipoic acid (LA) has been shown to elevate levels of glutathione through transcriptional processes. c. N-acetylcysteine (NAC) is an established synthetic precursor to glutathione. Administration of N-acetylcysteine will allow for elevated synthesis of glutathione. The combination of CA and MA and LA and/or N-acetylcysteine provides a dietary nutraceutical intervention for age-related neurodegeneration and neurodegenerative disease. Elevating neuronal glutathione improves endogenous redox defense, decreases nSMase-mediated production of ceramides, and combats symptoms of age-related neurodegeneration.

Administration of malic acid and citric acid decreases the pro-inflammatory and proapoptotic nSMase-mediated ceramide response to inflammatory cytokine tumor necrosis alpha (TNF-α) in humans. Elevated sensitivity of nSMase and elevated neuronal ceramides are strongly associated with neurodegeneration, decreased learning and memory associated with aging and neurodegeneration, inhibited mitochondrial function, and decreased insulin sensitivity. Supplementation with an effective amount of MA and CA will mitigate these problems.

Supplementation of MA and CA has not been previously linked to neuroprotection or improving learning and memory in aging or neurodegenerative models. Thus, MA and CA can reduce ceramide-mediated neurodegeneration, improves learning and memory, and improves neuronal mitochondrial function and insulin sensitivity.

Administration of LA and the concomitant elevation of glutathione decreases redox stress, restores nSMase-mediated ceramide balance, reduces mitochondrial decay, and modulates insulin sensitivity. Additionally, supplementation of LA has reduced memory loss in old rats and therefore can be considered a putative treatment for Alzheimer's-related dementia.

N-acetylcysteine is the most bioavailable precursor to glutathione. Supplementation of N-acetylcysteine elevates cellular levels of glutathione and has demonstrated the ability to reduce neuronal oxidative stress, inhibit ceramide production, and delay aging-associated loss of memory.

The classic approach to coping with redox stress through dietary supplementation is the use of antioxidative compounds which directly scavenge free radicals. High levels of dietary anti-oxidants have been shown to promote pro-oxidant effects, limiting the effectiveness of this approach. Disclosed herein is an alternative technology to increase the body's endogenous redox defense network.

Existing pharmaceutical interventions for nSMase inhibition are global and non-selective. The compositions and methods disclosed herein provide for normal, healthy apoptotic responses, but inhibits nSMase mediated ceramide production due to sensitized nSMase in the aging or diseased brain and tempers the response due to low-level perfuse inflammation exacerbated by reduced glutathione.

### C. Compositions

Disclosed herein is a composition comprising a) at least about 50 mg of citric acid; b) at least about 50 mg of malic acid; and c) at least about 100 mg of N-acetylcysteine, or at least about 100 mg of α-lipoic acid, or a mixture thereof. In one aspect, the composition comprises a pharmaceutically acceptable carrier.

In one aspect, a dosage form comprising the composition is intended for once daily dosing. In another aspect, a dosage form comprising the composition is intended for twice daily dosing.

In one aspect, the composition comprises an amount of citric acid that is therapeutically effective. In a further aspect the composition comprises an amount of malic acid that is therapeutically effective. In a further aspect, the composition comprises an amount of α-lipoic acid that is therapeutically effective. In a further aspect, the composition comprises an amount of N-acetylcysteine that is therapeutically effective.

Therapeutically effective refers to an amount that is sufficient to achieve the desired result or to have an effect on an undesired condition. In this sense, therapeutically effective means to provide a pharmacological impact or modulation of a subject wherein a disease or condition is cured or improved. The pharmacological responses elicited by the disclosed composition include but are not limited to improving learning, improving memory, improving cognitive ability, improving neuronal insulin sensitivity, increasing glutathione levels, decreasing neuronal ceramide levels or decreasing the activity of neuronal sphingomyeliase (nSMase).

In one aspect, the composition comprises an amount of citric acid that is prophylactically effective. In a further aspect the composition comprises an amount of malic acid that is prophylactically effective. In a further aspect, the composition comprises an amount of α-lipoic acid that is prophylactically effective. In a further aspect, the composition comprises an amount of N-acetylcysteine that is prophylactically effective.

In one aspect, a dosage form comprising the composition has a final weight suitable for administration to a subject, wherein the final weight is less than 25 g. In a further aspect, a dosage form comprising the composition has a final weight suitable for administration to a subject, wherein the final weight is less than 15 g. In a further aspect, a dosage form comprising the composition has a final weight suitable for administration to a subject, wherein the final weight is less than 10 g. In a further aspect, a dosage form comprising the composition has a final weight suitable for administration to a subject, wherein the final weight is less than 9 g. In a further aspect, a dosage form comprising the composition has a final weight suitable for administration to a subject, wherein the final weight is less than 8 g. In a further aspect, a dosage form comprising the composition has a final weight suitable for administration to a subject, wherein the final weight is less than 7.5 g. In a further aspect, a dosage form comprising the composition has a final weight suitable for administration to a subject, wherein the final weight is less than 7 g. In a further aspect, a dosage form comprising the composition has a final weight suitable for administration to a subject, wherein the final weight is less than 6.5 g. In a further aspect, a dosage form comprising the composition has a final weight suitable for administration to a subject, wherein the final weight is less than 6 g. In a further aspect, a dosage form comprising the composition has a final weight suitable for administration to a subject, wherein the final weight is less than 5.5 g. In a further aspect, a dosage form comprising the composition has a final weight suitable for administration to a subject, wherein the final weight is less than 5 g. In a further aspect, a dosage form comprising the composition has a final weight suitable for administration to a subject, wherein the final weight is less than 4.5 g. In a further aspect, a dosage form comprising the composition has a final weight suitable for administration to a subject, wherein the final weight is less than 4 g. In a further aspect, a dosage form comprising the composition has a final weight suitable for administration to a subject, wherein the final weight is less than 3.5 g. In a further aspect, a dosage form comprising the composition has a final weight suitable for administration to a subject, wherein the final weight is less than 3 g. In a further aspect, a dosage form comprising the composition has a final weight suitable for administration to a subject, wherein the final weight is less than 2.5 g. In a further aspect, a dosage form comprising the composition has a final weight suitable for administration to a subject, wherein the final weight is less than 2 g. In a further aspect, a dosage form comprising the composition has a final weight suitable for administration to a subject, wherein the final weight is less than 1.5 g. In a further aspect, a dosage form comprising the composition has a final weight suitable for administration to a subject, wherein the final weight is less than 1 g.

Disclosed herein is a composition comprising a) at least about 500 mg of citric acid; b) at least about 500 mg malic acid; c) at least about 100 mg N-acetylcysteine; and d) at least about 100 mg α-lipoic acid.

Disclosed herein is a composition comprising a) citric acid in an amount greater than 10 wt. %; b) malic acid in an amount greater than 10 wt. %; c) N-acetylcysteine in an amount greater than 1 wt. %; and d) α-lipoic acid in an amount greater than 0.5 wt. %.

Also disclosed herein is a composition comprising at least about 500 mg of citric acid and malic acid, wherein both citric acid and malic acid are in amounts greater than about 25 mg. In a further aspect, the composition comprises greater than about 100 mg of N-acetylcysteine or greater than about 100 mg of α-lipoic acid or a mixture thereof.

1. Citric Acid

In one aspect, the amount of citric acid is between 500-20,000 mg. In another aspect, the amount of citric acid is between 500-8,000 mg. In another aspect, the amount of citric acid is between 500-7,000 mg. In another aspect, the amount of citric acid is between 300-5,000 mg. In another aspect, the amount of citric acid is between 500-4,000 mg. In another aspect, the amount of citric acid is between 500-3,000 mg. In another aspect, the amount of citric acid is between 500-2,000 mg. In another aspect, the amount of citric acid is between 1,000-5,000 mg.

In one aspect, the composition comprises citric acid in an amount greater than 500 mg. In another aspect, the composition comprises citric acid in an amount greater than 2,500 mg. In another aspect, the composition comprises citric acid in an amount greater than 5,000 mg. In another aspect, the composition comprises citric acid in an amount greater than 9,000 mg.

In one aspect, the amount of citric acid is about 10 wt. % to about 60 wt. %. In another aspect, the amount of citric acid is about 15 wt. % to about 50 wt. %. In another aspect, the amount of citric acid is about 20 wt. % to about 45 wt. %. In another aspect, the amount of citric acid is about 30 wt. % to about 40 wt. %.

In one aspect, the amount of citric acid is greater than 10 wt. %. In another aspect, the amount of citric acid is greater than 20 wt. %. In another aspect, the amount of citric acid is greater than 30 wt. %. In another aspect, the amount of citric acid is greater than 40 wt. %. In another aspect, the amount of the citric acid is greater than 50 wt. %.

2. Malic Acid

In one aspect, the amount of malic acid is between 500-20,000 mg. In another aspect, the amount of malic acid is between 500-8,000 mg. In another aspect, the amount of malic acid is between 500-7,000 mg. In another aspect, the amount of malic acid is between 500-5,000 mg. In another aspect, the amount of malic acid is between 400-4,000 mg. In another aspect, the amount of malic acid is between 500-3,000 mg. In another aspect, the amount of malic acid is between 500-2,000 mg. In another aspect, the amount of malic acid is between 1,000-5,000 mg.

In one aspect, the composition comprises malic acid in an amount greater than 500 mg. In another aspect, the composition comprises malic acid in an amount greater than 2,500 mg. In another aspect, the composition comprises malic acid in an amount greater than 5,000 mg. In another aspect, the composition comprises malic acid in an amount greater than 9,000 mg.

In one aspect, the amount of malic acid is about 10 wt. % to about 60 wt. %. In another aspect, the amount of malic acid is about 15 wt. % to about 50 wt. %. In another aspect, the amount of malic acid is about 20 wt. % to about 45 wt. %. In another aspect, the amount of malic acid is about 30 wt. % to about 40 wt. %.

In one aspect, the amount of malic acid is greater than 10 wt. %. In another aspect, the amount of malic acid is greater than 20 wt. %. In another aspect, the amount of malic acid is greater than 30 wt. %. In another aspect, the amount of malic acid is greater than 40 wt. %. In another aspect, the amount of malic acid is greater than 50 wt. %.

3. α-Lipoic Acid

In one aspect, the amount of α-lipoic acid is between 100-5,000 mg. In another aspect, the amount of α-lipoic acid is between 150-4,000 mg. In another aspect, the amount of α-lipoic acid is between 200-3,000 mg. In another aspect, the amount of α-lipoic acid is between 300-2,000 mg. In another aspect, the amount of α-lipoic acid is between 400-1,000 mg. In another aspect, the amount of α-lipoic acid is between 500-900 mg. In another aspect, the amount of α-lipoic acid is between 600-700 mg.

In one aspect, the composition comprises α-lipoic acid in an amount greater than 300. In another aspect, the composition comprises α-lipoic acid in an amount greater than 600 mg. In another aspect, the composition comprises α-lipoic acid in an amount greater than 800 mg. In another aspect, the composition comprises α-lipoic acid in an amount greater than 900 mg.

In one aspect, the amount of α-lipoic acid is about 0.5 wt. % to about 50 wt. % of an administered dose. In another aspect, the amount of α-lipoic acid is about 5 wt. % to about 40 wt. % of an administered dose. In another aspect, the amount of α-lipoic acid is about 10 wt. % to about 30 wt. % of an administered dose. In another aspect, the amount of α-lipoic acid is about 15 wt. % to about 20 wt. % of an administered dose.

In one aspect, the amount of α-lipoic acid is greater than 0.5 wt. %. In another aspect, the amount of α-lipoic acid is greater than 10 wt. %. In another aspect, the amount of α-lipoic acid is greater than 15 wt. %. In another aspect, the amount of α-lipoic acid is greater than 25 wt. %.

4. N-acetylcysteine

In one aspect, the amount of N-acetylcysteine is between 100-5,000 mg. In another aspect, the amount of N-acetylcysteine is between 150-4,000 mg. In another aspect, the amount of N-acetylcysteine is between 200-3,000 mg. In another aspect, the amount of N-acetylcysteine is between 300-2,000 mg. In another aspect, the amount of N-acetylcysteine is between 400-1,000 mg. In another aspect, the amount of N-acetylcysteine is between 500-900 mg. In another aspect, the amount of N-acetylcysteine is between 600-700 mg.

In one aspect, the composition comprises N-acetylcysteine in an amount greater than 500 mg. In another aspect, the composition comprises N-acetylcysteine in an amount greater than 600 mg. In another aspect, the composition comprises N-acetylcysteine in an amount greater than 1,000 mg. In another aspect, the composition comprises N-acetylcysteine in an amount greater than 1,500 mg. In another aspect, the composition comprises α-lipoic acid in an amount greater than 300 mg and N-acetylcysteine in an amount greater than 500 mg. In another aspect, the composition comprises α-lipoic acid in an amount greater than 600 mg and N-acetylcysteine in an amount greater than 600 mg. In another aspect, the composition comprises α-lipoic acid in an amount greater than 800 mg and N-acetylcysteine in an amount greater than 1,000 mg. In another aspect, the composition comprises α-lipoic acid in an amount greater than 900 mg and N-acetylcysteine in an amount greater than 1,500 mg.

In one aspect, the amount of N-acetylcysteine is about 0.5 wt. % to about 50 wt. % of an administered dose. In another aspect, the amount of N-acetylcysteine is about 5 wt. % to about 40 wt. % of an administered dose. In another aspect, the amount of N-acetylcysteine is about 10 wt. % to about 30 wt. % of an administered dose. In another aspect, the amount of N-acetylcysteine is about 15 wt. % to about 20 wt. % of an administered dose.

In one aspect, the amount of N-acetylcysteine is greater than 0.5%. In another aspect, the amount of N-acetylcysteine is greater than 10%. In another aspect, the amount of N-acetylcysteine is greater than 15%. In another aspect, the amount of N-acetylcysteine is greater than 25%.

5. Composition Uses and Additional Elements

In a one aspect, the composition is a dietary supplement, wherein a dietary supplement is a compound or composition that is intended to compensate the dietary intake of a subject with regard to specific nutrients, vitamins, minerals, or other dietary components that may be missing or deficient in the subject's normal diet.

In one aspect, the composition is a pharmaceutical composition, wherein a pharmaceutical composition is a compound or composition that is administered to a subject to improve a therapeutic endpoint. A pharmaceutical composition may comprise the composition in combination with a pharmaceutically acceptable carrier or diluent.

In one aspect, the composition is useful as a medicament. In a further aspect, a medicament comprises the composition in combination with a pharmaceutically acceptable carrier or diluent.

In one aspect, the composition is useful in treating neurodegeneration. In a further aspect, neurodegeneration includes any kind of neurodegeneration including but not limited to Alzheimer's Disease, Parkinson's Disease, Huntington's Disease, Amyotrophic lateral sclerosis, age-related neurodegeneration, obesity-related neurodegeneration, diabetes-related neurodegeneration, TBI-related neurodegeneration, stroke-related neurodegeneration and non-alcoholic steatohepatitis-related neurodegeneration.

In one aspect, the composition contains at least one anti-oxidant agent, wherein an anti-oxidant agent refers to a compound, composition, or molecule that is capable of scavenging free radicals, reducing reactive oxygen species or halting oxidative stress chain reactions. Anti-oxidant agents known in the art include but are not limited to non-flavonoid antioxidants and nutrients that can directly scavenge free radicals including multicarotenes, beta-carotenes, alpha-carotenes, gamma-carotenes, lycopene, lutein and zeaxanthins, selenium, Vitamin E, including alpha-, beta- and gamma- (tocopherol, particularly alphatocopherol, etc., vitamin E succinate, and trolox (a soluble Vitamin E analog) Vitamin C (ascorbic acid) and Niacin (Vitamin B3, nicotinic acid and nicotinamide), Vitamin A, 13-cis retinoic acid, N-acetyl-L-cysteine, sodium ascorbate, pyrrolidin-edithio-carbamate, and coenzyme Q₁₀; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates O₂H₂O₂, glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavenoids and flavenoid like molecules like folic acid and folate, or a combination thereof.

In one aspect, the composition contains at least one compound that increases neuronal glutathione, inhibits activation of neuronal sphingomyelinase (nSMase), or decreases neuronal ceramides. Compounds known in the art to increase levels of glutathione include but are not limited to N-acetylcysteine, S-adenosyl-methionine (SAM), ornithine decaroxylase (OTC) and oxothiazolidine carboxylate (OTZ), glutathione monoesters and glutathione diesters, L-cysteine, L-methionine, melatonin, glutamine, lipoic acid, silymarin, and whey proteins, or a combination thereof.

In one aspect, the composition is suitable for administering to a subject. In a further aspect, the subject is a mammal. In a further aspect, the subject is a human.

In one aspect, the composition is non-toxic with respect to humans.

In one aspect, the compositions disclosed herein can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Routes of administration are dependent on the particular form of the composition. Routes of administration include but are not limited to orally administered, buccally administered, nasally administered, topically administered, sublingually administered, subcutaneously administered, parenterally administered, rectally administered, intravaginally administered, intravenously administered, or intramuscularly administered.

It is understood that the disclosed compositions can be prepared from the disclosed compounds. It is also understood that the disclosed compositions can be employed in the disclosed methods of using.

### D. Methods of Using Compositions

In one aspect, disclosed herein is a method for treating a neurodegenerative disorder associated with glutathione dysregulation in a subject comprising the step of administering to the subject an effective amount the disclosed compositions, or a mixture thereof, thereby treating a neurodegenerative disorder associated with glutathione dysregulation. Treating a neurodegenerative disorder can include treating symptoms associated with a neurodegenerative disorder. Furthermore, treating a neurodegenerative disorder includes decreasing the progression of a neurodegenerative disorder, such as the progression of the early stages of the neurodegenerative disorder.

Neurodegenerative disorders associated with glutathione dysregulation include but are not limited to Alzheimer's Disease, Parkinson's Disease, Huntington's Disease, Amyotrophic lateral sclerosis and age-related neurodegeneration.

In one aspect, disclosed herein is a method for decreasing the rate of neurodegeneration in a subject comprising the step of administering to the subject an effective amount of the disclosed composition, thereby decreasing the rate of neurodegeneration in the subject.

Neurodegeneration is generally a progressive, irreversible condition. As neurodegeneration progresses, the subject beings losing cognitive abilities and motor skills including but not limited to the ability learn and remember things, and the ability to move or feed oneself, respectively. Neurodegeneration can progress into dementia and ataxia, leaving a subject unable to completely take care of themselves. The rate at which these symptoms develop can have a dramatic impact on the quality of life of an individual suffering from a neurodegenerative disorder as well as the family of the individual. Thus, slowing the rate of neurodegeneration is preferable.

Also disclosed herein is a method for enhancing the memory in a subject comprising administering to the subject an effective amount of the disclosed composition, thereby enhancing the memory in the subject. In one aspect, enhancing memory can be the recovery of cognitive functions lost due to aging, TBI, and/or stroke.

Also disclosed herein is a method for decreasing the rate of neurodegeneration and improving learning and memory in a subject comprising administering to the subject an effective amount of the disclosed composition, thereby decreasing the rate of neurodegeneration and improving the learning and memory in the subject. The neurodegeneration can be neurodegeneration due to aging, TBI, and/or stroke. The improving of learning and memory can be recovery of cognitive functions lost due aging, TBI, and/or stroke.

Also disclosed herein is a method for increasing glutathione, inhibiting the activation of nSMase and reducing levels of neuronal ceramides in at least one cell comprising contacting the cell with an effective amount of the disclosed composition, thereby increasing glutathione, inhibiting the activation of nSMase and reducing levels of neuronal ceramides in the at least one cell.

In one aspect, the cell is mammalian. In another aspect, the cell is human. In another aspect, the cell has been isolated from a mammal prior to the contacting step. In another aspect, contacting is via administration to a mammal In another aspect, contacting is performed in vitro. In another aspect, contacting is performed in vivo.

Also disclosed herein is a method for reducing ceramide mediated neurodegeneration in a subject comprising administering to the subject an effective amount of the disclosed composition, thereby reducing ceramide mediated neurodegeneration in the subject.

Ceramide mediated neurodegeneration refers to an umbrella of conditions including but not limited to Alzheimer's Disease, Parkinson's Disease, Huntington's Disease, Amyotrophic lateral sclerosis, age-related neurodegeneration, obesity-related neurodegeneration, diabetes-related neurodegeneration, TBI-related neurodegeneration, stroke-related neurodegeneration, and non-alcoholic steatohepatitis-related neurodegeneration.

Also disclosed herein is a method for reducing ceramide-mediated neurodegeneration, improving learning and memory, and improving neuronal mitochondrial function and insulin sensitivity in a subject comprising administering to the subject an effective amount of the disclosed composition, thereby reducing ceramide-mediated neurodegeneration, improving learning and memory, and improving neuronal mitochondrial function and insulin sensitivity in the subject.

Also disclosed herein is a method for improving learning in a subject comprising administering to the subject an effective amount of the disclosed composition, thereby improving learning in the subject.

Also disclosed herein is a method for improving neuronal mitochondrial function in a subject comprising administering to the subject an effective amount of the disclosed composition, thereby improving neuronal mitochondrial function in the subject.

Also disclosed herein is a method for improving insulin sensitivity in a subject comprising administering to the subject an effective amount of the disclosed composition, thereby improving insulin sensitivity in the subject.

In one aspect, an effective amount is a therapeutically effective amount. In a further aspect, the subject is a human. In a further aspect, glutathione dysregulation is associated with lower than normal levels of glutathione in a subject. In a further aspect, the subject is in need of treatment for a neurodegenerative disorder associated with glutathione dysregulation. In a further aspect, the neurodegenerative disease is related to aging. In a further aspect, the subject has been diagnosed with a neurodegenerative disease associated with glutathione dysregulation prior to the step of administration. In a further aspect, the subject has been diagnosed with a neurodegenerative disease associated with glutathione dysregulation. In a further aspect, the method further comprises the step of identifying a subject in need of treatment of the disorder.

In another aspect, the subject is in need of decreasing the rate of neurodegradation. In another aspect, the subject has been diagnosed with an abnormal rate of neurodegradation. In another aspect, the method further comprises the step of identifying a subject in need of decreasing the rate of neurodegradation. In another aspect, rate of neurodegeneration is associated with glutathione dysregulation. In another aspect, the rate of neurodegeneration is associated with glutathione dysregulation is age-related.

In another aspect, the subject is in need of enhancing memory. In another aspect, the subject has been diagnosed with an abnormal memory. In another aspect, the method further comprises the step of identifying a subject in need of enhancing memory. In another aspect, the enhancing of memory is a statistically significant enhancement in a test of learning and memory including, but not limited to, Wisconsin Card Sorting Test, the California Verbal Learning Test, the Wechsler Memory Scale-Revised, the Visual Reproduction subscale, or other tests well known to those skilled in the art (Solomon, P. R. 2002. JAMA. 288(7):835-840).

In another aspect, the subject is in need of reducing ceramide mediated neurodegeneration. In another aspect, the subject has been diagnosed with a need of reducing ceramide mediated neurodegeneration. In another aspect, the method further comprises the step of identifying a subject in need of reducing ceramide mediated neurodegeneration.

In another aspect, the subject is in need of reducing ceramide-mediated neurodegeneration, improving learning and memory, and improving neuronal mitochondrial function and insulin sensitivity. In another aspect, the subject has been diagnosed with a need of reducing ceramide-mediated neurodegeneration, improving learning and memory, and improving neuronal mitochondrial function and insulin sensitivity. In another aspect, the method further comprises the step of identifying a subject in need of reducing ceramide-mediated neurodegeneration, improving learning and memory, and improving neuronal mitochondrial function and insulin sensitivity.

In another aspect, the subject is in need of improving learning. In another aspect, the subject has been diagnosed with a need of improving learning. In another aspect, the method further comprises the step of identifying a subject in need of improving learning.

In another aspect, the subject is in need of improving neuronal mitochondrial function. In another aspect, the subject has been diagnosed with a need of improving neuronal mitochondrial function. In another aspect, the method further comprises the step of identifying a subject in need of improving neuronal mitochondrial function.

In another aspect, the subject is in need of improving insulin sensitivity. In another aspect, the subject has been diagnosed with a need of improving neuronal mitochondrial function. In another aspect, the method further comprises the step of identifying a subject in need of improving insulin sensitivity.

The amount of citric acid and malic acid administered to the subject in the methods disclosed herein can be dependent on the bodyweight of the subject. In one aspect, the methods disclosed herein can comprise administering up to about 200 mg of citric acid per kilogram of bodyweight of the subject. For example, the methods disclosed herein can comprise administering up to about 175 mg, 150 mg, 125 mg, 100 mg, 75 mg, 50 mg or 25 mg of citric acid per kilogram of bodyweight of the subject. In another aspect, the methods disclosed herein can comprise administering up to about 200 mg of malic acid per kilogram of bodyweight of the subject. For example, the methods disclosed herein can comprise administering up to about 175 mg, 150 mg, 125 mg, 100 mg, 75 mg, 50 mg or 25 mg of malic acid per kilogram of bodyweight of the subject.

The amount of N-acetylcysteine, or α-lipoic acid, or a mixture thereof administered to the subject in the methods disclosed herein can be dependent on the bodyweight of the subject. In one aspect, the methods disclosed herein can comprise administering up to about 100 mg of N-acetylcysteine and/or α-lipoic acid per kilogram of bodyweight of the subject. For example, the methods disclosed herein can comprise administering up to about 75 mg, 50 mg, 25 mg, or 10 mg of N-acetylcysteine and/or α-lipoic acid per kilogram of bodyweight of the subject.

### E. Kits

Also disclosed herein is a kit comprising a composition comprising the disclosed composition. In another aspect, the kit further comprises at least one agent known to increase glutathione. In another aspect, the kit further comprises at least one agent known to inhibit activation of nSMase. In another aspect, the kit further comprises at least one agent known to decrease the levels of neuronal ceramides. In another aspect, the kit further comprises instructions for treating a disorder associated with glutathione dysregulation. In another aspect, the kit further comprises instructions for treating a disorder associated with nSMase dysregulation. In another aspect, the kit further comprises instructions for treating a disorder associated with altered levels of neuronal ceramides. In another aspect, the kit further comprises an agent known to decrease glutathione. In another aspect, the kit further comprises an agent known to stimulate activation of nSMase. In another aspect, the kit further comprises an agent known to increase levels of neuronal ceramides. In another aspect, the kit comprises the composition co-formulated with at least one agent disclosed herein. In another aspect the kit comprises the composition co-packaged with at least one agent disclosed herein.

### F. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

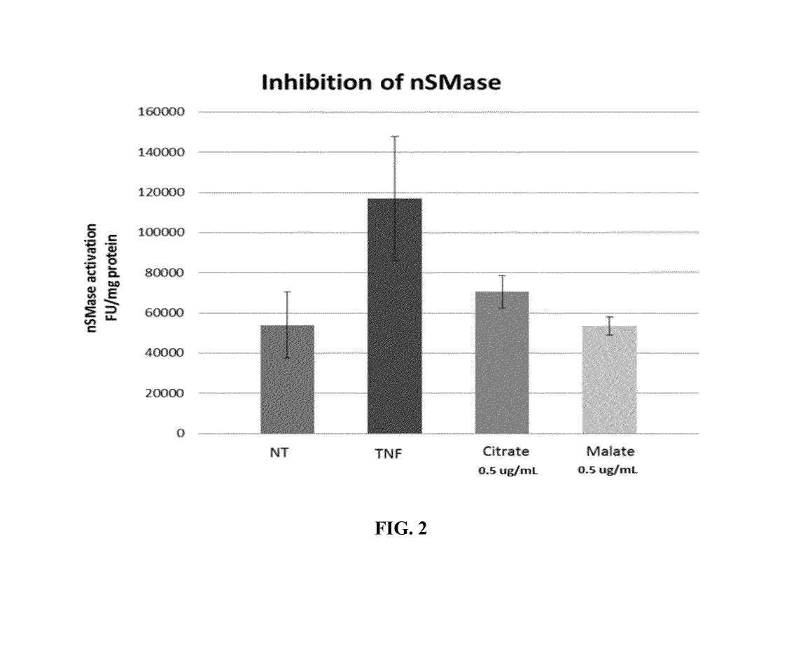

Citrate/malate treatments have been shown to significantly decrease neutral sphingomyelinase (nSMase)-mediated ceramide release in tumor necrosis factor (TNFα)-treated neuroblastomas, as shown in FIG. 2. Ceramides and ceramide metabolites are important intracellular signaling molecules which influence inflammation, proliferative and apoptotic processes, neurodegeneration, and insulin sensitivity. Elevated ceramides are associated with neurodegenerative disorders including Alzheimer's and Parkinson's disease as well as normal aging. The following data provide chemical and biological rationale for the observed inhibition of nSMase and decreased ceramide production via increased levels of glutathione (GSH). The data additionally provides evidence for the role of citrate and malate in endogenous redox defense and neuroprotection.

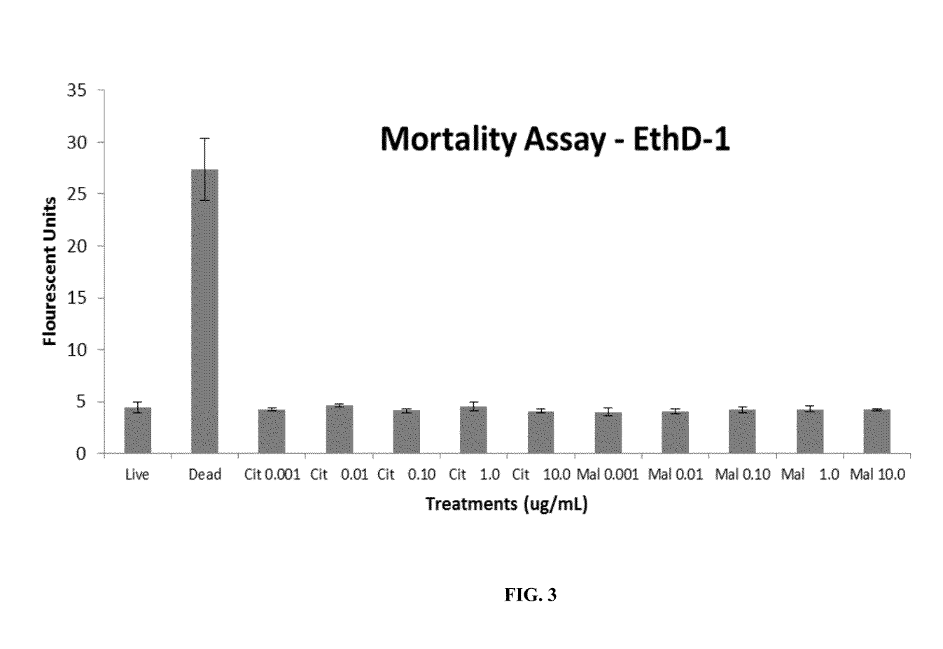

Aqueous preparations of citrate and malate demonstrate no acute cytotoxic effects on model neurons at biologically relevant concentrations. SH-SY5Y human neuroblastomes were plated in black 96 well tissue culture well plates at a seeding density of 10,000 cells per well. Neurons were grown for 24 hours in DMEM supplemented with 10% FBS, then treated with supplemented DMEM including either citrate or malate, with concentrations ranging from 0.001-10.0 μg/mL, for 24 hours. Following 24 hour incubation, samples were rinsed twice with PBS and incubated with ethidium homodimer (5 μM in PBS) for 1 hour. Cytotoxic response was evaluated by red fluorescent response of ethidium homodimer with a fluorescent microplate reader. Results of these assays are shown in FIG. 3. Total cytotoxic response was compared to non-treated neurons and a positive control for cell death (100% MeOH for 30 minutes). Additionally, 24 aged rats were orally administered 200 mg/Kg malate through voluntary consumption in water, and no ill effects were observed by the researchers, support staff or veterinarian.

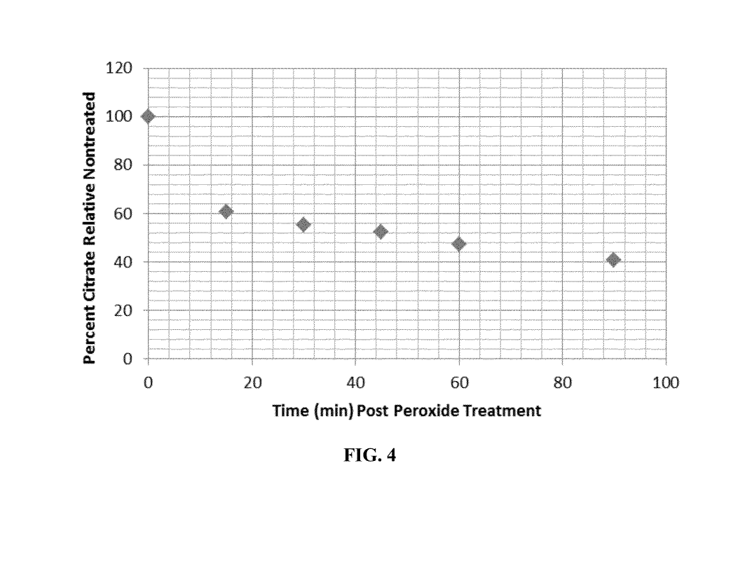

Intracellular concentrations of total citrate clearly decrease in response to oxidative insult, indicating citrate is actively consumed as part of the endogenous redox defense response in human neurons, specifically to produce NADPH. As shown in FIG. 4, intracellular citrate decreases 40% in the first 15 minutes following peroxide treatment and continues to slowly decrease over the rest of the time course. SH-SY5Y human neuroblastoma cells were grown to 90% confluency on 100 mm tissue culture dishes in 10% FBS supplemented DMEM, serum starved for 12 hours, and treated with 100 μl H₂O₂ in serum free DMEM for either 0, 15, 30, 45, 60, or 90 minutes. All treatments were performed in triplicate. Following treatment, neurons were rinsed with PBS, harvested and evaluated for total citrate via abcam citrate assay kit (ab83396). Briefly, citrate is converted to pyruvate via oxaloacetate. The pyruvate is then converted to an intensely fluorescent product.

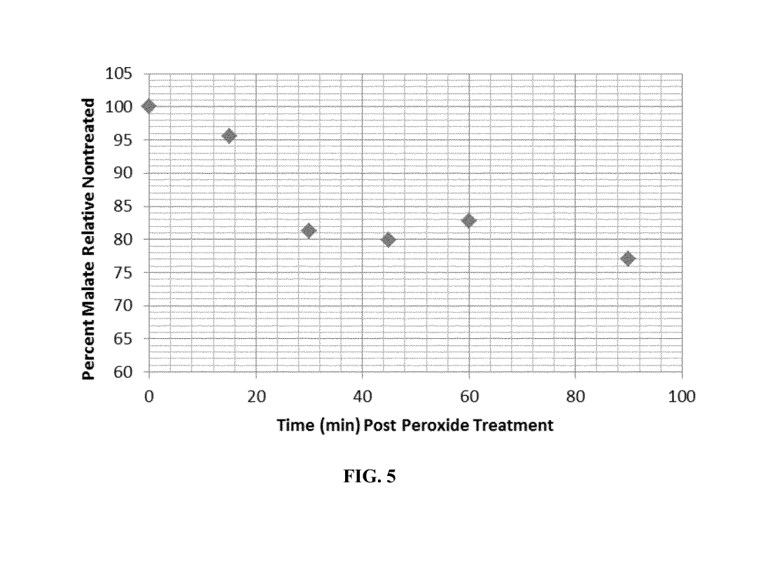

Intracellular concentrations of total malate clearly decrease in response to oxidative insult, as shown in FIG. 5, indicating malate is actively consumed as part of the endogenous redox defense response in human neurons, specifically to produce NADPH. Intracellular malate decreases 20% in the first 30 minutes following peroxide treatment and continues to slowly decrease over the rest of the time course. SH-SY5Y human neuroblastoma cells were grown to 90% confluency on 100 mm tissue culture dishes in 10% FBS supplemented DMEM, serum starved for 12 hours, and treated with 100 μM H₂O₂ in serum free DMEM for either 0, 15, 30, 45, 60, or 90 minutes. All treatments were performed in triplicate. Following treatment, neurons were rinsed with PBS, harvested and evaluated for total citrate via abcam malate assay kit (ab83391). Briefly, malate is specifically oxidized to generate a product which reacts with a substrate probe to produce a colored product.

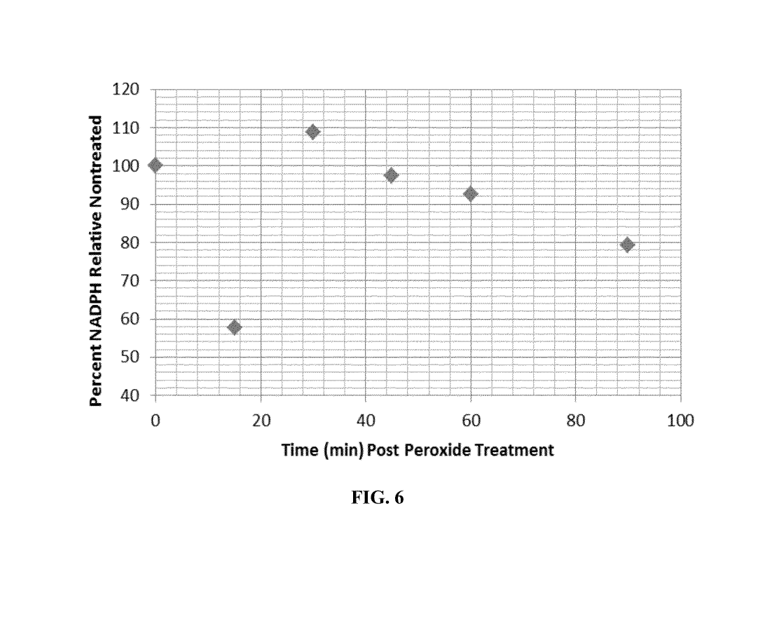

Intracellular concentrations of total NADPH transiently decrease in response to oxidative insult, indicating NADPH is actively consumed as part of the endogenous redox defense response in human neurons, specifically to regenerate GSH. As shown in FIG. 6, intracellular NADPH decreases 42% in the first 15 minutes following peroxide treatment and quickly rebounds at the 30 minute mark due to secondary pathways (pentose phosphate shunt). Intracellular NADPH continues to decline under sustained redox stress for the remaining course of the experiment. SH-SY5Y human neuroblastomes were plated to black 96 well tissue culture well plates at a seeding density of 10,000 cells per well and grown in 10% FBS supplemented DMEM for 24 hours, serum starved for 12 hours, and treated with 100 μM H₂O₂ in serum free DMEM for either 0, 15, 30, 45, 60, or 90 minutes. All treatments were performed in triplicate. Following treatment, neurons were rinsed with PBS, harvested and evaluated for total NADPH via eEnzyme NADPH assay kit.

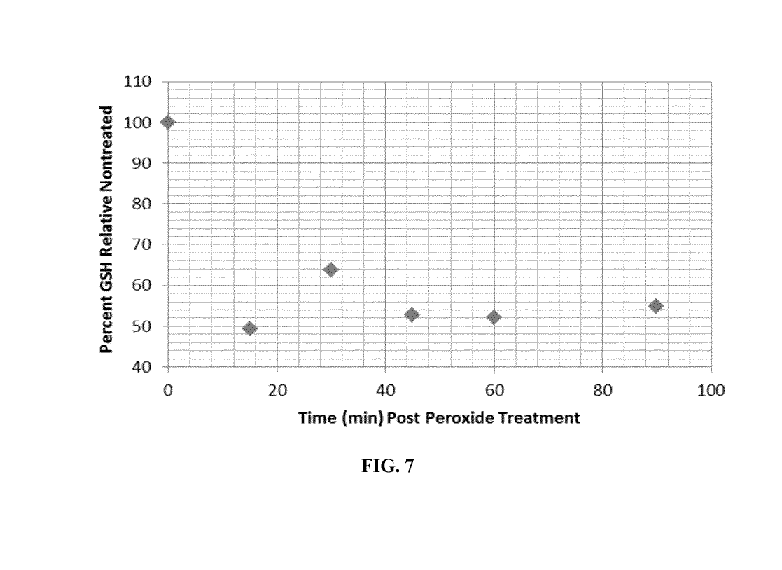

GSH is widely considered the cornerstone of endogenous redox defense. Intracellular concentrations of total GSH clearly decrease in response to oxidative insult, confirming GSH is actively consumed as part of the endogenous redox defense response in human neurons, specifically to produce regenerate antioxidative enzymes. As shown in FIG. 7, intracellular GSH decreases 50% in the first 15 minutes following peroxide treatment, show a slight rebound at 30 minutes and subsequently sustain levels between 50-60% of non-treated neurons. SH-SY5Y human neuroblastomes were plated to white 96 well tissue culture well plates at a seeding density of 10,000 cells per well and grown in 10% FBS supplemented DMEM for 24 hours, serum starved for 12 hours, and treated with 100 μm H₂O₂ in serum free DMEM for either 0, 15, 30, 45, 60, or 90 minutes. All treatments were performed in triplicate. Following treatment, neurons were rinsed with PBS and evaluated for total GSH (excluding GSSG) via Promega GSH-Glo Glutathione Assay. The assay is based on the conversion of a lucifin derivative into lucifierin in the presence of GSH.

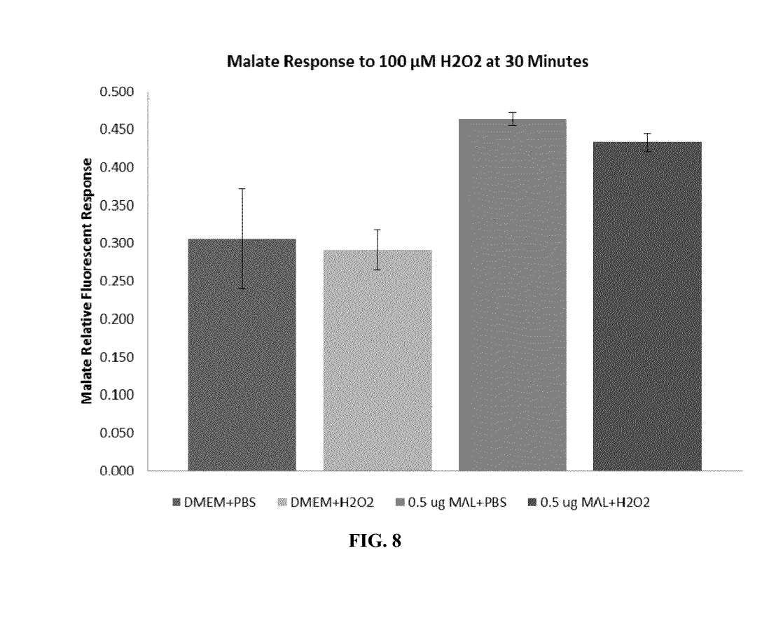

Pretreatment of SH-SY5Y human neuroblastoma cells with malate increase intracellular pools of malate and prevent critical depletion of malate, as shown in FIG. 8. This indicates that malate supplementation is an effective means of maintaining working pools of this critical component of neuronal redox defense. Cells were treated with 0.5 μg/mL malate in serum free DMEM for 1 hour prior to peroxide insult. Malate supplementation increased intracellular malate by nearly 50%. Even after peroxide insult, malate-supplemented cells demonstrated higher levels than non-treated and non-insulted cells.

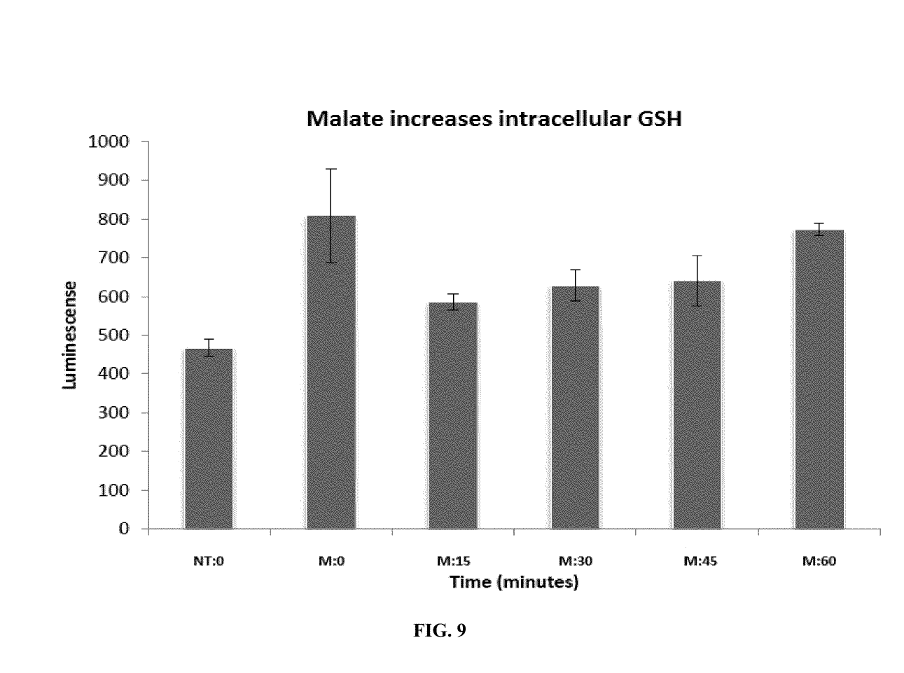

Pretreatment of SH-SY5Y human neuroblastoma cells with malate increase intracellular pools of GSH and prevent critical depletion of GSH following redox insult. This indicates that malate supplementation is an effective means of maintaining working pools of GSH for neuronal redox defense. Cells were treated with 0.5 μg/mL malate in serum free DMEM for 1 hour prior to peroxide insult. Intracellular GSH was determined at 0, 15, 30, 45, and 60 minutes, post peroxide, as shown in FIG. 9. Malate supplemented cells show significantly higher levels of GSH than non-supplemented cells, and even after peroxide insult, malate-supplemented cells demonstrated higher levels of GSH than non-treated and non-insulted cells.

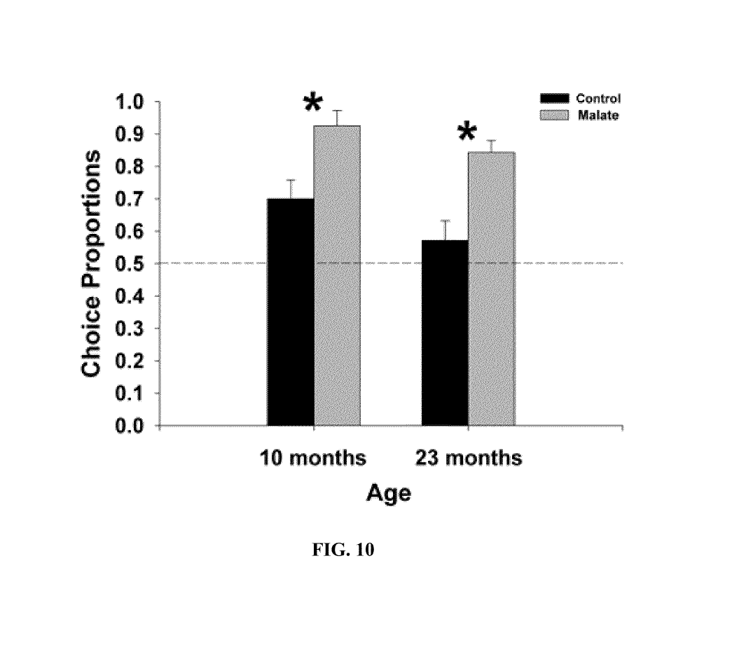

Aged rats provided with 200 mg/Kg malate in water demonstrated statistically significant improvement in spatial memory over control rats not provided supplemental malate. 24 male Fischer F344 inbred rats were evaluated at 10 months and 23 months of age. Half were provided supplemental dietary malate. Following 6 weeks supplementation, spatial memory was evaluated using a rewarded alternation procedure in a T-maze apparatus (10 trials per subject). Healthy, non-impaired, rats alternate at 85% while hippocampectomized rats incapable of encoding spatial memories alternate at chance, 50%. The 10 month old, control, rats demonstrated impaired spatial memory, alternating at 70%, while malate supplemented rats alternated at 92.5%. The 23 month old, control, rats were severely impaired, hardly scoring better than chance, at 57% alternation. 23 month old malate-supplemented rats alternated at 85%, a score equivalent to a young, not impaired rat. (FIG. 10). These data demonstrate that dietary malate is a potent intervention for preventing and treating aging-related neurodegeneration and memory impairment.

The data presented show that metabolism of citrate and malate is an essential source of NADPH production in redox insulted neuroblastoma cells and that depletion of citrate and malate during periods of redox stress leads to reduced pools of GSH and subsequent activation of nSMase. The metabolic relationship between citrate, malate, NADPH, GSH, nSMase, ceramide production and redox stress are summarized graphically above.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### G. References

The following references, to the extent that they provide exemplary procedural or other details supplementary to those set forth herein, are specifically incorporated herein by reference in their entirety.
1. Allen, C L. and Bayrakutan, U. Oxidative stress and its role in the pathogenesis of ischaemic stroke. 2009. Int J Stroke, 4(6); 461-470
2. Anderson, J K. Oxidative stress in neurodegeneration: cause or consequence? Jul. 1, 2004. Nature Reviews Neuroscience, 5; S18-S25
3. Barth BM. et al. Neutral sphingomyelinase activation precedes NADPH oxidase-dependent damage in neurons exposed to the proinflammatory cytokine tumor necrosis factor-α. 2012. J. Neurosci Res. 90(1); 229-242
4. Fukui K. et al. Impairment of learning and memory in rats caused by oxidative stress and aging, and changes in antioxidative defense systems. 2001. Ann N Y Acad Sci, 928: 168-175
5. Pearce, R K. et al. Alterations in the distribution of glutathione in the substantia nigra in Parkinson's disease. 1997. J. Neural Transm. 104, 661-677
6. Riederer, P. et al. Transition metals, ferrentin, glutathione, and ascorbic acid in pakinsonianneutra brains. 1989. J. Neurochem. 52, 515-520
7. Rodriquez-Rodriquez, A. et al. Oxidative Stress In Traumatic Brain Injury. 2013. Curr Med Chem, Epub ahead of print
8. Solomon, P. R. et al. 2002. Ginko for memory enhancement: A randomized controlled trial. JAMA. 288(7):835-840
9. Tong M. et al. Mechanisms of ceramide-mediated neurodegeneration. 2009. J Alzheimers Dis. 16(4); 705-714
10. Urano S. et al. Aging and oxidative stress in neurodegeneration. 1998. Biofactors, 7(1-2); 103-112

What is claimed is:

1. A composition comprising
a) at least about 500 mg of citric acid;
b) at least about 500 mg of malic acid; and
c) at least about 100 mg of N-acetylcysteine, or at least about 100 mg of α-lipoic acid, or a mixture thereof.

2. The composition of claim 1, wherein the composition further comprises a pharmaceutically acceptable carrier.

3. The composition of claim 1, wherein the composition comprises
a) at least about 500 mg of citric acid;
b) at least about 500 mg of malic acid;
c) at least about 100 mg of N-acetylcysteine; and
d) at least about 100 mg of α-lipoic acid.

4. The composition of claim 1, wherein the citric acid is about 1,000 mg to about 5,000 mg.

5. The composition of claim 1, wherein the malic acid is about 1,000 mg to about 5,000 mg.

6. The composition of claim 1, wherein the composition comprises greater than about 300 mg of α-lipoic acid.

7. The composition of claim 1, wherein the composition comprises greater than about 500 mg of N-acetylcysteine.

8. The composition of claim 1, wherein citric acid is about 10 wt. % to about 60 wt. %, wherein wt. % is based on the total weight of the formulation or composition in which the component is included.

9. The composition of claim 1, wherein malic acid is about 10 wt. % to about 60 wt. %, wherein wt. % is based on the total weight of the formulation or composition in which the component is included.

10. The composition of claim 1, wherein α-lipoic acid is about 0.5 wt. % to about 50 wt. %, wherein wt. % is based on the total weight of the formulation or composition in which the component is included.

11. The composition of claim 1, wherein N-acetylcysteine is about 0.5 wt. % to about 50 wt. %, wherein wt. % is based on the total weight of the formulation or composition in which the component is included.

12. The composition of claim 1, wherein the composition further comprises a compound that increases neuronal glutathione, inhibits activation of neuronal sphingomyelinase (nSMase), or decreases neuronal ceramides.

13. A kit comprising the composition of claim 1, and one or more of:
a) at least one agent known to increase glutathione;
b) at least one agent known to inhibit activation of nSMase;
c) at least one agent known to decrease the levels of neuronal ceramides;
d) instructions for treating a disorder associated with glutathione dysregulation;
e) instructions for treating a disorder associated with nSMase dysregulation;
f) instructions for treating a disorder associated with altered levels of neuronal ceramides;
g) an agent known to decrease glutathione;
h) an agent known to stimulate activation of nSMase; and
i) an agent known to increase levels of neuronal ceramides.

14. A method for treating a neurodegenerative disorder associated with glutathione dysregulation in a subject comprising the step of administering to the subject a therapeutically effective amount of a composition comprising
a) at least about 500 mg of citric acid;
b) at least about 500 mg of malic acid; and
c) at least about 100 mg of N-acetylcysteine, or at least about 100 mg of α-lipoic acid, or a mixture thereof,
thereby treating the neurodegenerative disorder associated with glutathione dysregulation.

15. The method of claim 14, wherein the subject is a human.

16. The method of claim 14, wherein the neurodegenerative disorder is selected from a group consisting of Alzheimer's Disease, Parkinson's Disease, Huntington's Disease, and Amyotrophic lateral sclerosis.

17. The method of claim 14, wherein the neurodegenerative disorder is related to aging.

18. A method for decreasing the rate of neurodegeneration in a subject comprising the step of administering to the subject a therapeutically effective amount of a composition comprising
a) at least about 500 mg of citric acid;
b) at least about 500 mg of malic acid; and
c) at least about 100 mg of N-acetylcysteine, or at least about 100 mg of α-lipoic acid or a mixture thereof,
thereby decreasing the rate of neurodegeneration in the subject.

19. The method of claim 18, wherein the subject is a human

20. The method of claim 18, wherein the subject is in need of decreasing the rate of neurodegradation.
